(19) United States
(12) Patent Application Publication
(10) Pub. No.: US 2011/0269850 A1
(43) Pub. Date: Nov. 3, 2011

(54) SHELLAC ENTERIC COATINGS

(76) Inventors: Charles A. Signorino, Norristown, PA (US); Terry L. Smith, Norristown, PA (US); Stephen Levine, Norristown, PA (US)

(21) Appl. No.: 12/769,645

(22) Filed: Apr. 28, 2010

(51) Int. Cl. A61K 47/46

(52) U.S. Cl. 514/778

(57) ABSTRACT

The field of the invention generally relates to a shellac-based enteric coating with a reliable and consistent dissolution profile in both acid and neutral environments based on the selective choice of coating excipients used with the shellac. The enteric coating formulation includes a shellac, a maltrin, sodium alginate, one or more plasticizers, and optionally talc. The shellac is present in a range of approximately 40% to 60% w/w % and has an acid number of between 71 and 73. The maltrin is present in a range of approximately 15% to 30% w/w %. The sodium alginate is present in a range of approximately 15% to 30% w/w %. The one or more plasticizers are present in a range of approximately 2% to 20% w/w %. The optional talc is present in a range of 0% to 10%.

### TECHNICAL FIELD

The field of the invention generally relates to a shellac-based enteric coating with a reliable and consistent dissolution profile in both acid and neutral environments based on the selective choice of coating excipients used with the shellac.

### BACKGROUND

There is a need to enterically coat tablets in the pharmaceutical and the nutraceutical industries. These coatings ensure that the active ingredient is not released in the acidic environment of the stomach but instead in the more neutral environment of the intestine. Some coatings are difficult to use on a large scale, often times as a result of their viscosity or another handling characteristic or preparation characteristic. Other coatings provide an inconsistent or unreliable result release profile.

Shellac is a known enteric coating material. It is well known in the art that shellac is an excellent film former and has been used for tablet coating for a century. Shellac films give good performance when freshly deposited out of alcohol but they crosslink and esterify in the dried film and extend the release characteristics after six months or a year. This particular defect has led to reduction in the use of shellac for tablet coating. The FDA lists the excipients used in NDA products every four years, in 1988 shellac was used in 278 listings; in 1992, 219 listings; in 1996 it was used in only 73 listings. Because of the problems with shellac its use has been declining for more than 30 years. Many patents and other references note shellac, but it is not promoted and instead is only mentioned as a known and historically used material. The pharmaceutical industry has moved away from shellac as a significant resin for coating. Forty years ago it was the most popular resin for enteric coating; today it is hardly used. The invention disclosed herein relates to new methods and formulations configured to cause shellac to be more reliable and give consistent results.

U.S. Pat. No. 5,567,438 discloses shellac coatings in which the shellac is dissolved in a basic aqueous solution to which is added an acidic aqueous solution to form a shellac suspension. The patent further discloses that a plasticizer, such as polyethylene glycol and polypropylene glycol, can be used in the coating formulation.

U.S. 20050031775, the contents of which are incorporated herein, discloses coating systems that include a shellac in an aqueous solution, a hydrolyzed starch product, and a plasticizer. Examples of the hydrolyzed starch product include maltodextrins with a dextrose equivalent (DE) greater than 10 and dried glucose syrups with a DE greater than 20. Examples of the plasticizer include polyethylene glycol and propylene glycol.

U.S. Pat. No. 6,620,431 discloses film coatings for enteric and colonic release at selected pH based on selecting and/or formulating a shellac of a predetermined acid number. The method disclosed includes the selection of the acid number to provide the release at the specified pH and a method of providing a shellac of a predetermined acid number by blending of shellacs of different acid numbers. The film coatings are described as including a water soluble resin and/or a plasticizer. The water soluble resins listed are hydroxypropyl methylcellulose (HPMC), methylcellulose (MC), hydroxypropylcellulose (HPC), polyvinyl pyrrolidone (PVP), modified starch or maltodextrin and natural gums such as acacia. The plasticizer is disclosed as being a fatty acid or a water soluble plasticizer. The fatty acid may be oleic acid, stearic acid or other fatty acids. The water soluble plasticizer may be glycerine, propylene glycol, polyethylene glycol (molecular weight 300-8000), triacetin (TA) and triethyl citrate.

### SUMMARY

In one general aspect, an enteric coating formulation includes a shellac, a maltrin, sodium alginate, one or more plasticizers, and optionally talc. The shellac is present in a range of approximately 40% to 60% w/w % and has an acid number of between 68 and 75. The maltrin is present in a range of approximately 15% to 30% w/w %. The sodium alginate is present in a range of approximately 15% to 30% w/w %. The one or more plasticizers are present in a range of approximately 2% to 20% w/w %. The optional talc is present in a range of 0% to 10%.

Embodiments of the enteric coating formulation may include one or more of the following features. For example, the plasticizer may be one or both of polyethylene glycol and glycerine. The coating may be configured to be applied at a range of approximately 2% to 10% weight gain of the coated dosage form. The coating, when applied to a solid dosage form, may be configured to prevent release of an active ingredient for at least one hour in a 1.2 pH buffer solution. The coating, when applied to a solid dosage form, may be configured to disintegrate in a 6.8 pH buffer solution in less than one hour. The shellac may have an acid number of approximately 71.

In another general aspect, an enteric coating formulation includes shellac, maltrin, sodium alginate, and glycerine. The shellac is present in a range of approximately 40% to 60% w/w % and has an acid number of between 68 and 75. The maltrin is present in a range of approximately 15% to 30% w/w %. The sodium alginate is present in a range of approximately 15% to 30% w/w %. The glycerine is present in a range of approximately 2% to 20% w/w %. The coating formulation may optionally include silica or talc in a range of 0% to 10%

Embodiments of the coating may include one or more of the following features. For example, the maltrin, sodium alginate and glycerine may be provided as a separate component from the shellac. The enteric coating formulation may further include a glycerine absorbing or adsorbing material in an amount sufficient to cause the maltrin, sodium alginate and glycerine to be a free flowing powder. The glycerine absorbing or adsorbing material may be present at between 1% and 10% by weight of the formulation.

In another general aspect, a free flowing powder includes maltrin, sodium alginate, glycerine and a glycerine adsorbing or absorbing material. The maltrin is present in a range of approximately 30% to 60% w/w %. The sodium alginate is present in a range of approximately 30% to 60% w/w %. The glycerine is present in a range of approximately 4% to 40% w/w %. The glycerine absorbing or adsorbing material is present in an amount sufficient to cause the maltrin, sodium alginate and glycerine to be a free flowing powder.

Embodiments of the free flowing powder may include one or more of the following features. For example, the glycerine absorbing or adsorbing material may be present at between 2% and 20% by weight of the formulation. The free flowing powder may further include a shellac as a separate component from the free flowing powder.

In another general aspect a method of forming an enteric coating includes: providing a dry powder component comprising maltrin, sodium alginate, glycerine and a glycerine adsorbing or absorbing material; providing a shellac; and adding water and shellac to the dry power component to form an enteric coating solution.

Embodiments of the method may include one or more of the following features. For example, the shellac may be present in a range of approximately 40% to 60% w/w %; the maltrin may be present in a range of approximately 15% to 30% w/w %; the sodium alginate may be present in a range of approximately 15% to 30% w/w %; the glycerine may be present in a range of approximately 2% to 20% w/w %; and the glycerine absorbing or adsorbing material may be present in a range of 1% to 10%.

In another general aspect, a method of forming an enteric coating includes: providing a dry powder component comprising maltrin, sodium alginate, and a plasticizer; providing a shellac; and adding water and shellac to the dry power component to form an enteric coating solution.

Embodiments of the method may include one or more of the following features. For example, the shellac may be present in a range of approximately 40% to 60% w/w %; the maltrin may be present in a range of approximately 15% to 30% w/w %; the sodium alginate may be present in a range of approximately 15% to 30% w/w %; and the plasticizer may be present in a range of approximately 2% to 20% w/w %. The method further include providing one or more of talc, silica, pigments, dyes and flavors in a range of 1% to 10%.

The details of various embodiments of the invention are set forth in the accompanying drawings and the description below. Other features and advantages of the invention will be apparent from the description, the drawings, and the claims.

### DETAILED DESCRIPTION

The inventors have developed shellac-based enteric coating formulations that have a reliable and consistent dissolution profile in acidic and neutral environments that mimic that of the gastro-intestinal tract. Specifically, the formulations are designed to be applied to a solid dosage form, last for at least one hour in a 1.2 pH buffer solution and disintegrate in a 6.8 pH buffer solution in less than one hour. The formulations can be all-natural formulations by using all natural ingredients to make the coating. In general, the formulations may have the following formula: Shellac (40-60%), maltrin (15-30%), sodium alginate (15-30%), plasticizer (2-20%) and optionally talc (0-10%). A variety of plasticizers, such as polyethylene glycol (PEG) and the natural plasticizer, glycerine, and alternate soluble resins in addition to maltrin may be used. Coatings in the range of 2-10 weight gain of the coated dosage form are expected to be suitable.

More specifically, the inventors have developed an all-natural enteric shellac coating that uses the natural ingredient, glycerine, as the plasticizer. An all natural coating is useful in a variety of fields, especially the nutraceutical and natural products markets. A suitable, all-natural formulation using glycerine can have the following general formula: Shellac (40-60%), maltrin (15-30%), sodium alginate (15-30%), glycerine (2-20%). While this all natural formulation is suitable for its intended purpose, for a commercially marketed product it is desirable to respond to the market need of convenience. A formulation designed for the convenience needed in manufacturing may be a two part kit, a shellac component and a dry excipient component. The dry excipient component can be a powder blend of the maltin, sodium alginate and glycerine. These can be mixed with water and then mixed with the shellac. Advantageously, such a kit has a long shelf life.

To be an even more convenient, all natural shellac coating, the inventors have discovered that a glycerine absorbing or adsorbing material should be used to provide a free flowing powder that is easily used at a manufacturing scale. The dry powder formulation without a glycerine absorbing or adsorbing material may form lumps or solids masses. While these materials can be used without glycerine, they are not conveniently used in large scale manufacturing. Instead, it is desirable to have a free flowing powder, which is provided by the glycerine absorbing or adsorbing material. The glycerine is absorbed into the material or adsorbed onto the surface of the material and thereby can be used to provide a free flowing powder. In this formulation, the glycerine absorbing or adsorbing material is mixed with the glycerine or with the glycerine and maltrin. The sodium alginate then is added to the mixture. The resulting mixture that includes the normally liquid glycerine becomes a dry mixture that is free flowing.

Examples of the glycerine absorbing or adsorbing material may be materials such as silica, talc, pigments, titanium dioxide, microcrystalline cellulose, lakes, iron oxide pigments, calcium carbonate, and the like. The glycerine absorbing or adsorbing material is selected based on its ability to be used at a low amount and provide a free flow powder that includes glycerine. While glycerine in particular has been described as the liquid plasticizer, other liquid plasticizers may be used in the formulation and a material that absorbs or adsorbs the liquid plasticizer used to cause a free flowing powder.

One suitable, free flowing, all-natural shellac enteric coating formulation using glycerine as the plasticizer is the following: shellac (50%) (as a first, separate component), maltrin (40%), sodium alginate (40%), glycerine (10%), talc (6%) and silica (4%) (combined as a second, separate component). In this example formulation, the free flowing powder of the formulation makes up 50% by weight of the coating and is made up of the maltrin, sodium alginate, glycerine, talc and silica. The percentages given above for maltrin, sodium alginate, glycerine, talc and silica relate to the weight percentages with respect to the dry power blend of those excipients (i.e., not including the weight of the shellac). For the percentages of the components of the coating as a whole, i.e., including the shellac, the percentages of each of the excipients of the second component would be divided in half. Thus, for the coating as a whole, the formulation would be shellac (50%), maltrin (20%), sodium alginate (20%), glycerine (50%), talc (3%) and silica (2%) In this formulation it is believed that the sodium alginate lowers the release pH of the film, the glycerine and maltrin further stabilize and facilitate the release of the coating.

It also should be understood that a plasticizer, such as polyethylene glycol, that is not a liquid, will not need a liquid plasticizer absorbing or adsorbing material to provide a convenient, free flowing powder. It also should be noted that in the kit described above, the dry powder component can include polyethylene glycol in place of glycerine.

Shellac is graded on its acid number, which is a measure of the number of carboxylic acid groups on the shellac polymer. There are some generalities known about shellac as a function of acid number. First, the higher the acid number, the lower the pH at which dissolution of the shellac takes place. Shellac with acid numbers 68-73 dissolves at pH above 7.4. Shellac with acid numbers 74-80 dissolves at pH 7 and above. Shellac with acid numbers above 80 dissolves at pH less than 7. Second, a lower acid number provides an increased stability of the shellac while a higher acid number provides a reduced stability of the shellac. The shellac used in the coating examples below is Marcoat 125™ available from Emerson Resources in Norristown, Pa. It has an acid number of 71 and is available as an aqueous solution containing 25% shellac. Consequently, it can be said that the aqueous solution contains 25% solids. Some shellacs are refined in a bleaching process with sodium hypochlorite. This often results in an acid number of approximately 90 or greater. Marcoat 125™ shellac, in contrast, is refined in a solvent extraction and charcoal decolorization process that does not modify the chemical structure of the shellac and provides an acid number of approximately 68 to 75 and more particularly approximately 71. The shellac is in the form of the ammonium salt of shellac.

The inventors have discovered that they surprisingly can go beyond what they disclosed in U.S. Pat. No. 6,620,431 and are now able to formulate a shellac with an acid number of approximately 71 to release at a pH of 6.8 by the inclusion of maltrin and sodium alginate. The inclusion of maltrin and sodium alginate modify the shellac film to make the film soluble at a lower than expected pH, but still resist dissolution in the gastric fluids having a pH of 1.2.

A first coating formulation is provided below in Table 1. The coating composition consists of a shellac (Marcoat 125™), dextrin (Maltrin 150), a plasticizer (Polyethylene glycol—PEG 3350), and sodium alginate.

TABLE 1
(TS79-071)

| | Grams/ | | Solids | Grams solid | % solids |
|---|---|---|---|---|---|
| Ingredient | batch | % w/w | (%) | per batch | w/w |
| Marcoat 125 ™ | 136.780 | 16.509 | 25 | 34.20 | 51.60 |
| Maltrin 150 | 11.270 | 1.360 | 100 | 11.27 | 17.00 |
| PEG 3350 | 8.750 | 1.056 | 100 | 8.75 | 13.20 |
| Sodium Alginate | 12.060 | 1.456 | 100 | 12.06 | 18.20 |
| Deionized water | 659.640 | 79.619 | 0 | 0 | 0.00 |
| Total Coating | 828.500 | 100.00 | 8.00 | 66.28 | 100.00 |
| Blend | | | | | |

The coating was formulated and then coated on softgel tablets in a 15 inch pan coater using the settings provided in Table 2. The coating used 1.5 kg of softgels on which the coating material was coated. In this system, 441 grams of coating were applied for a 2% weight gain, 485 grams of coating applied for a 2.2% weight gain, and 551 grams for a 2.5% weight gain. The coating solution had a pH of 7.15.

TABLE 2

| | | Intake | Exhaust | Air Flow | | | Grams |
|---|---|---|---|---|---|---|---|
| Time | Weight | Temp | Temp | Blower | Atom | | per |
| (min) | (grams) | (° C.) | (° C.) | Setting | (psi) | RPM | minute |
| 0 | 0 | 55.0 | 34.8 | 253 | 48.2 | 18.0 | — |
| 5 | 59 | 54.6 | 33.8 | 244 | 47.9 | 18.0 | 11.8 |
| 10 | 126 | 54.2 | 34.3 | 246 | 47.5 | 18.0 | 13.4 |
| 15 | 202 | 54.8 | 36.2 | 257 | 47.6 | 18.0 | 15.2 |
| 20 | 276 | 55.2 | 37.3 | 248 | 47.3 | 18.0 | 14.8 |
| 30 | 420 | 55.4 | 38.5 | 256 | 47.2 | 18.0 | 14.4 |
| 39.3 | 551 | 55.0 | 39.4 | 253 | 47.2 | 18.0 | 14.1 |

The coated softgel tablets next were subjected to disintegration testing in both gastric and intestinal mediums of 1.2 pH and 6.8 pH, respectively. Three tablets were placed in each of three baths of pH 1.2, the tablets varying by coating weight gain. Each bath consisted of 900 ml of fluid, was kept at 37±2° C., and the coated softgels remained in the bath for one hour. In the first bath the three tablets tested were coated to a weight gain of 2.0%, in the second bath the three tablets tested were coated to a weight gain of 2.2%, and in the third bath the three tablets tested were coated to a weight gain of 2.5%.

The tablets from the pH 1.2 testing next were placed in one of three baths of approximately pH 6.8, the tablets separated according to coating weight gain. Each bath consisted of 900 ml of fluid, was kept at 37±2° C., and the coated softgels remained in the bath for one hour. There was a slight variation in the pH of the three baths. In the first bath at a 6.87 pH the three tablets tested were coated to a weight gain of 2.0%, in the second bath at a pH 6.87 pH the three tablets tested were coated to a weight gain of 2.2%, and in the third bath at a pH 6.92 the three tablets tested were coated to a weight gain of 2.5%. The tablets were tested for disintegration based on end softgels opening up and releasing the contents of the softgels. The tablets with a 2.0% weight gain had an average disintegration time of 15.1 minutes, the tablets with a 2.2% weight gain had an average disintegration time of 17.7 minutes and the tablets with a 2.5% weight gain had an average disintegration time of 20.0 minutes.

A repeat of the testing of the coated batch of tablets showed that two of three of the 2.0% weight gain softgel tablets disintegrated at pH 1.2. As in the testing described above, the softgel tablets of weight gain 2.2% and 2.5% did not disintegrate at pH 1.2. The coated softgel tablets were tested at pH 6.87 with the following results: the tablets with a 2.2% weight gain had an average disintegration time of 18.7 minutes and the tablets with a 2.5% weight gain had an average disintegration time of 21.7 minutes. Because two of three of the tablets tested at pH 1.2 disintegrated, the 2.0% weight gain softgel tablets were not tested at the higher pH.

A second batch of coating solution having the formulation of Table 1 with 8% solids was produced and its viscosity tested at 25° C. over a revolution per minute range of 30 to 100 RPM. The viscosity ranged between 120.0 cP and 123.0 cP with an error range for the readings ranging between 113.0 cP and 133.0 cP. The inventors believe that the viscosity values in this range are all suitable for a workable coating formulation.

A second coating formulation was developed that differed from the formulation of Table 1 by the use of 10% solids rather than the 8% solids of the formulation illustrated in Table 1. The 10% solids formulation is illustrated below in Table 3.

TABLE 3

| | | | | Grams | |
|---|---|---|---|---|---|
| | Grams/ | | Solids | solid | % solids |
| Ingredient | batch | % w/w | (%) | per batch | w/w |
| Marcoat 125 ™ | 206.400 | 20.640 | 25 | 51.60 | 51.60 |
| Maltrin 150 | 17.000 | 1.700 | 100 | 17.00 | 17.00 |
| PEG 3350 | 13.200 | 1.320 | 100 | 13.20 | 13.20 |
| Sodium Alginate | 18.200 | 1.820 | 100 | 18.20 | 18.20 |
| Deionized water | 745.200 | 74.520 | 0 | 0 | 0.00 |
| Total Coating | 1000.000 | 100.00 | 10.00 | 100.00 | 100.00 |
| Blend | | | | | |

The coating formulation of Table 3 was tested in the same conditions as the formulation above. The viscosity measured had ranged between 261.0 and 290.0 with an error range for the readings ranging between 258.0 cP and 300.0 cP.

A third coating formulation was developing using gylcerine in place of polyethylene glycol 3350 as the plasticizer. The formulation is illustrated in Table 4.

TABLE 4

| | | | | Grams | |
|---|---|---|---|---|---|
| | Grams/ | | Solids | solid | % solids |
| Ingredient | batch | % w/w | (%) | per batch | w/w |
| Marcoat 125 ™ | 165.120 | 16.512 | 25 | 41.28 | 51.60 |
| Maltrin 150 | 14.560 | 1.456 | 100 | 14.56 | 18.20 |
| Glycerine | 9.600 | 0.960 | 100 | 9.60 | 12.00 |
| Sodium Alginate | 14.560 | 1.456 | 100 | 14.56 | 18.20 |
| Deionized water | 796.160 | 79.616 | 0 | 0 | 0.00 |
| Total Coating | 1000.000 | 100.00 | 8.00 | 80.00 | 100.00 |
| Blend | | | | | |

The coating was formulated and then coated on softgel tablets in a 15 inch pan coater using the settings provided in Table 5. The coating used 1.5 kg of softgels on which the coating material was coated. In this system, 551 grams of coating were applied for a 2.5% weight gain, 662 grams of coating applied for a 3.0% weight gain, and 772 grams for a 3.5% weight gain. The coating solution had a pH of 7.12.

TABLE 5

| | | Intake | Exhaust | Air Flow | | | Grams |
|---|---|---|---|---|---|---|---|
| Time | Weight | Temp | Temp | Blower | Atom | | per |
| (min) | (grams) | (° C.) | (° C.) | Setting | (psi) | RPM | minute |
| 0 | 0 | 64.9 | 38.2 | 251 | 48.1 | 18.0 | — |
| 5 | 61 | 59.9 | 37.1 | 255 | 47.9 | 18.0 | 12.2 |
| 10 | 169 | 54.2 | 37.9 | 247 | 47.6 | 18.0 | 10.8 |
| 20 | 233 | 53.0 | 37.0 | 245 | 47.4 | 18.0 | 12.8 |
| 30 | 359 | 57.5 | 38.2 | 251 | 47.4 | 18.0 | 12.6 |
| 40 | 486 | 53.9 | 39.5 | 254 | 47.1 | 18.0 | 12.7 |
| 50 | 548 | 51.8 | 39.2 | 244 | 47.0 | 18.0 | 12.4 |
| 60 | 672 | 52.0 | 39.5 | 247 | 47.2 | 18.0 | 12.4 |
| 68 | 772 | 52.0 | 39.6 | 256 | 47.2 | 18.0 | 12.4 |

The formulation of Table 4 with 8% solids was produced and its viscosity tested at 25° C. over a revolution per minute range of 30 to 100 RPM. The viscosity ranged between 111.0 cP and 113.0 cP with an error range for the readings ranging between 103.0 cP and 123.0 cP. The inventors believe that the viscosity values in this range are all suitable for a workable coating formulation.

The coated softgel tablets next were subjected to disintegration testing in both gastric and intestinal mediums of 1.2 pH and 6.8 pH, respectively. Three tablets were placed in each of three baths of pH 1.2, the tablets varying by coating weight gain. Each bath consisted of 900 ml of fluid, was kept at 37±2° C., and the coated softgels remained in the bath for one hour. In the first bath the three tablets tested were coated to a weight gain of 2.5%, in the second bath the three tablets tested were coated to a weight gain of 3.0%, and in the third bath the three tablets tested were coated to a weight gain of 3.5%. The softgel tablets did not disintegrate.

The tablets from the pH 1.2 testing next were placed in one of three baths of approximately pH 6.8, the tablets separated according to coating weight gain. Each bath consisted of 900 ml of fluid, was kept at 37±2° C., and the coated softgels remained in the bath until the tablets were disintegrated. In the first bath the three tablets tested were coated to a weight gain of 2.5%, in the second bath the three tablets tested were coated to a weight gain of 3.0%, and in the third bath the three tablets tested were coated to a weight gain of 3.5%. The tablets were tested for disintegration based on end softgels opening up and releasing the contents of the softgels. The tablets with a 2.5% weight gain had an average disintegration time of 21.9 minutes, the tablets with a 3.0% weight gain had an average disintegration time of 27.9 minutes and the tablets with a 3.5% weight gain had an average disintegration time of 31.8 minutes.

While several particular forms of the invention have been illustrated and described, it will be apparent that various modifications and combinations of the invention detailed in the text and drawings can be made without departing from the spirit and scope of the invention. For example, references to materials of construction, methods of construction, specific dimensions, shapes, utilities or applications are also not intended to be limiting in any manner and other materials and dimensions could be substituted and remain within the spirit and scope of the invention. Accordingly, it is not intended that the invention be limited, except as by the appended claims.

What is claimed is:

1. An enteric coating formulation comprising:
a shellac being present in a range of approximately 40% to 60% w/w % and having an acid number of between 68 and 75;
a maltrin being present in a range of approximately 15% to 30% w/w %;
sodium alginate being present in a range of approximately 15% to 30% w/w %;
one or more plasticizers being present in a range of approximately 2% to 20% w/w %; and
optionally talc being present in a range of 0% to 10%

2. The enteric coating formulation of claim 1, wherein the plasticizer comprises one or both of polyethylene glycol and glycerine.

3. The enteric coating formulation of claim 1, wherein the coating is configured to be applied at a range of approximately 2% to 10% weight gain of the coated dosage form.

4. The enteric coating formulation of claim 1, wherein the coating, when applied to a solid dosage form, is configured to prevent release of an active ingredient for at least one hour in a 1.2 pH buffer solution.

5. The enteric coating formulation of claim 1, wherein the coating, when applied to a solid dosage form, is configured to disintegrate in a 6.8 pH buffer solution in less than one hour.

6. The enteric coating formulation of claim 1, wherein the shellac as an acid number of approximately 71.

7. An enteric coating formulation comprising:
shellac being present in a range of approximately 40% to 60% w/w % and having an acid number of between 68 and 73;
maltrin being present in a range of approximately 15% to 30% w/w %;
sodium alginate being present in a range of approximately 15% to 30% w/w %;
glycerine being present in a range of approximately 2% to 20% w/w %; and
optionally talc being present in a range of 0% to 10%

8. The enteric coating formulation of claim 7, wherein the maltrin, sodium alginate and glycerine are provided as a separate component from the shellac.

9. The enteric coating formulation of claim 7, further comprising a glycerine absorbing or adsorbing material in an amount sufficient to cause the maltrin, sodium alginate and glycerine to be a free flowing powder.

10. The enteric coating formulation of claim 9, wherein the glycerine absorbing or adsorbing material comprises between 1% and 10% by weight of the formulation.

11. A free flowing powder comprising:
a maltrin being present in a range of approximately 30% to 60% w/w %;
sodium alginate being present in a range of approximately 30% to 60% w/w %;
glycerine being present in a range of approximately 4% to 40% w/w %; and
a glycerine absorbing or adsorbing material in an amount sufficient to cause the maltrin, sodium alginate and glycerine to be a free flowing powder.

12. The free flowing powder of claim 11, wherein the glycerine absorbing or adsorbing material comprises between 2% and 20% by weight of the formulation.

13. The free flowing powder of claim 11, further comprising shellac as a separate component from the free flowing powder.

14. A method of forming an enteric coating, the method comprising:
providing a dry powder component comprising maltrin, sodium alginate, glycerine and a glycerine adsorbing or absorbing material;
providing a shellac; and
adding water and shellac to the dry power component to form an enteric coating solution.

15. The method of claim 14, wherein the shellac is present in a range of approximately 40% to 60% w/w %; the maltrin is present in a range of approximately 15% to 30% w/w %; the sodium alginate is present in a range of approximately 15% to 30% w/w %; the glycerine is present in a range of approximately 2% to 20% w/w %; and and the glycerine absorbing or adsorbing material is present in a range of 1% to 10%.

16. A method of forming an enteric coating, the method comprising:
providing a dry powder component comprising maltrin, sodium alginate, and a plasticizer;
providing a shellac; and
adding water and shellac to the dry power component to form an enteric coating solution.

17. The method of claim 16, wherein the shellac is present in a range of approximately 40% to 60% w/w %; the maltrin is present in a range of approximately 15% to 30% w/w %; the sodium alginate is present in a range of approximately 15% to 30% w/w %; and the plasticizer is present in a range of approximately 2% to 20% w/w %.

18. The method of claim 17, further comprising one or more of talc, silica, pigments, dyes and flavors present in a range of 1% to 10%.
